(19) United States
(12) Patent Application Publication
(10) Pub. No.: US 2026/0325436 A1
(43) Pub. Date: Oct. 22, 2026

(54) COSMETIC COMPOSITION COMPRISING PORTULACA OLERACEA EXTRACT

(71) Applicant: BEIERSDORF AG, Hamburg (DE)

(72) Inventors: Eduardo C. RUVOLO, Flanders, NJ (US); Donna HWANG, Leonia, NJ (US); Yongyi ZHANG, New Providence, NJ (US)

(21) Appl. No.: 19/181,631

(22) Filed: Apr. 17, 2025

(51) Int. Cl. A61K 8/9789; A61K 8/27; A61K 8/29; A61K 8/35; A61K 8/37; A61K 8/40; A61Q 17/04; A61Q 19/02

(52) U.S. Cl. CPC A61K 8/9789; A61K 8/27; A61K 8/29; A61K 8/35; A61K 8/37; A61K 8/40; A61Q 17/04; A61Q 19/02

(57) ABSTRACT

A topical cosmetic composition which comprises an extract of Portulaca oleracea in a concentration which is effective for reducing pigmentation and skin-darkening in melanin-rich skin.

### BACKGROUND OF THE INVENTION

### 1. Field of the Invention

The present invention relates to the use of an extract of Portulaca oleracea for reducing or preventing pigmentation and skin darkening in melanin-rich skin.

### 2. Discussion of Background Information

The human skin serves as the body's first line of defense against extrinsic aggressors such as UV radiation, pollution, and oxidative stress. Exposure to UV radiation is the most common, and also the most preventable, cause of skin cancers. Skin damage by exposure to UV radiation occurs through at least two mechanisms: (i) direct damage to DNA, mainly from UVB (280-320 nm), and (ii) production of free radicals from UVA (320-400 nm), which destroy intracellular components including DNA, membranes, and proteins. Cosmetic products with protection against UV are commonly marketed as gel, cream, emulsion, or spray and aim to protect the skin of a person from the radiation emitted by the sun.

However, existing sunscreen compositions have historically been designed for lighter skin types, often leaving visible residues, or inadequately addressing the needs of melanin-rich skin. Melanin-rich skin, commonly classified as Fitzpatrick skin types IV-VI, has unique physiological characteristics, including higher melanin content, which offers some natural protection against UV damage. Nonetheless, it is not immune to the harmful effects of UV radiation.

The key challenges faced by melanin-rich skin are: (i) hyperpigmentation and skin darkening where UV and visible light (VL) exposure can trigger excessive melanin production, leading to uneven skin tone and dark spots, (ii) post-inflammatory hyperpigmentation which causes the skin to discolor following UV and VL exposure or inflammation, and (iii) compatibility issues that lead to a visible white residue when applied at effective concentrations to melanin-rich skin.

In view of the foregoing, it would be advantageous to be able to provide extended protection of melanin-rich skin against damage caused by solar radiation, especially in order to suppress pigmentation and erythema induced by solar radiation in the UVB, UVA, and VL ranges.

Pigmentary changes caused by VL and UVA have been shown to occur in three phases, i.e., immediate pigment darkening (IPD) which is dose dependent and lasts up to 2 hours after irradiation, followed by persistent pigment darkening (PPD) that continues up to 24 hours, and lastly delayed tanning (DT) which occurs approximately 7 days after irradiation. Both IPD and PPD have been suggested to be a result of oxidation and redistribution of pre-existing melanin, whereas delayed tanning is exhibited by the formation of new melanin.

Despite the fact that VL and UVA have been shown to be associated with pigment darkening and worsening conditions such as post-inflammatory hyperpigmentation and melasma, there are only limited options for preventing these conditions available. Furthermore, there is a lack of formulations specifically tailored to melanin-rich skin, which requires products that not only provide broad-spectrum UV protection but also prevent skin darkening and promote skin health. Accordingly, there remains a need for more effective sunscreen compositions, and particularly compositions that can provide effective sunscreen protection while minimizing skin darkening and promote skin health for skin of color.

The present inventors have found that the problem of regulating the behavior of melanin-rich skin in response to solar radiation is complicated but very important, in particular when formulating products for application to melanin-rich skin with a view to ensuring a satisfactory protection against skin darkening and erythema. It is also desirable to increase the capacity of the skin to resist pigmentation in melanin-rich skin caused by solar radiation.

### SUMMARY OF THE INVENTION

The present invention provides a cosmetic composition which is formulated for topical application to skin and comprises at least one extract of Portulaca oleracea in a concentration which is effective for reducing pigmentation and skin-darkening in melanin-rich skin in response to solar radiation.

In one embodiment, the composition of the invention comprises from about 0.001% to about 0.5% by weight, e.g., from about 0.002% to about 0.03% by weight, of an extract of Portulaca oleracea, based on the total weight of the composition. For example, the composition may comprise at least 0.003%, at least 0.005% at least 0.01%, at least 0.02%, at least 0.05%, at least 0.08%, at least 0.1%, at least 0.15%, at least 0.20%, at least 0.25%, at least 0.30%, at least 0.35%, at least 0.40% or at least 0.45% by weight of an extract of Portulaca oleracea.

In one embodiment, the composition of the invention further comprises diethylhexyl syringylidene malonate. For example, the composition may comprise from about 0.1% to about 3% by weight of diethylhexyl syringylidene malonate, based on the total weight of the composition.

In one embodiment, the composition of the invention further comprises one or more organic and/or inorganic UV filter substances. For example, the composition may comprise from about 1% to about 50% by weight of the one or more UV filter substances, based on the total weight of the composition. Non-limiting examples of the UV filter substances which may be present in the composition of the invention include one or more of butyl methoxydibenzoylmethane, octocrylene, homosalate, ethylhexyl salicylate, zinc oxide, and titanium dioxide.

In one embodiment, the composition of the present invention further comprises at least one thickener and/or ethanol and/or glycyrrhetinic acid and/or Glycyyrhiza Inflata root extract.

In one embodiment, the composition of the invention is present as a gel, lotion, cream or spray.

In one embodiment, the composition of the invention an emulsion, e.g., an O/W emulsion or a W/O emulsion.

In one embodiment of the composition of the invention, at least one extract of Portulaca oleracea present therein has been obtained by extracting the entire plant of Portulaca oleracea or a part thereof with ethanol and/or water.

In one embodiment of the composition of the invention, the extract of Portulaca oleracea present therein is capable of reducing the pigmentation of melanin-rich skin and preventing skin darkening caused by UV radiation and visible light and/or is capable of alleviating or preventing post-inflammatory hyperpigmentation in melanin-rich skin and/or is capable of alleviating or preventing excessive melanin production triggered by UV radiation in melanin-rich skin.

The present invention further provides a method of reducing pigmentation and skin-darkening in melanin-rich skin. The method comprises topically applying to skin in need thereof the composition of the invention as set forth above (including the various embodiments thereof) in an amount which is effective for reducing pigmentation and skin-darkening in melanin-rich skin.

In one embodiment of the method of the invention, the method may be a therapeutic method. In another embodiment, the method may be a cosmetic or non-therapeutic method.

In one embodiment of the method of the invention, post-inflammatory hyperpigmentation in melanin-rich skin is alleviated or prevented.

In one embodiment of the method of the invention, the pigmentation of melanin-rich skin is reduced and skin darkening caused by UV radiation and visible light is reduced or prevented.

In one embodiment of the method of the invention, excessive melanin production triggered by UV radiation in melanin-rich skin is reduced or prevented.

The term “melanin-rich skin”, commonly classified as Fitzpatrick skin types IV-VI, is associated with unique physiological characteristics, including higher melanin content, which offers some natural protection against UV damage.

The term “solar radiation” preferably refers to solar electromagnetic radiation from 290 nm to 3.0 μm, preferably from 290 nm to 800 nm, most preferably from 290 nm to 500 nm. Such radiation can be emitted from natural sources such as sunlight as well as from artificial devices such as a solar simulator. More preferably, in all embodiments of the present invention, the term “solar radiation” refers to UVB, UVA and visible light.

The term “skin” as used herein refers to human skin.

The cosmetic composition of the present invention comprises an (at least one) extract of natural origin as an active ingredient.

In the present invention, “extract” means that the extract is separated and obtained from the active ingredient raw material using a method known in the art for extraction and separation, wherein the extract defined in the present invention is prepared by using an appropriate extraction solvent or solvent mixture.

The extract of Portulaca oleracea present in the composition of the present invention is an extract from the plant of Portulaca oleracea that is an annual succulent with bright yellow flowers and is an edible nutritious vegetable which is generally used in traditional Chinese and Korean medicines. Portulaca oleracea contains bioactive compounds such as antioxidants, omega-3 fatty acids, flavonoids, alkaloids, polysaccharides, and minerals.

The extract of Portulaca oleracea in the present invention may be obtained by extracting the plant or a part thereof with water, anhydrous or hydrous lower alcohols, polyhydric alcohols, hydrocarbon-based solvents, or mixture thereof. Specific examples include water, ethanol, glycerol, propanediol, and 1,2-hexanediol.

The ratio of extracted substances from the plant or parts thereof is dependent on, inter alia, the extraction solvent(s) used, the temperature, pressure and duration of extraction. Other sources of variation include steps taken to concentrate or remove targeted constituents or classes of constituents, and the compounds formed during extraction or further processing thereof. For example, ethanol and water mixtures can sometimes offer selectivity as the ratio of ethanol to water can be adjusted to target specific compound classes. A higher ethanol content may be more suitable for extracting flavonoids, while a lower ethanol content may be better for some alkaloids.

Further, the extraction rate of the active ingredients contained in the plant raw material may depend on the polarity of the solvent(s) used for the extraction.

As for the extraction method, any method such as hot water extraction method, cold extraction method, reflux cooling extraction method, solvent extraction method, steam distillation method, ultrasonic extraction method, elution method, compression method, etc. can be selected and used. There is no limitation on the method for preparing the extract for use in the composition of the present invention, and any known method may be used.

The effectiveness of the extract used in the present invention is dependent on inter alia, the extraction method and solvent used for extraction.

The extract of Portulaca oleracea may be used to enhance the functionality of a cosmetic composition, such as an antioxidant protection where the extract neutralizes reactive oxygen species (ROS), which can damage skin cells and accelerate aging.

The extract of Portulaca oleracea present in the composition of the invention may also be used for reducing inflammation and redness associated with UV exposure, sunburn, or environmental aggressors.

Further, the extract of Portulaca oleracea present in the composition of the invention may also help to alleviate post-inflammatory hyperpigmentation, a common problem in the case of melanin-rich skin.

The extract of Portulaca oleracea present in the composition of the invention may also be used to regulate melanin production via modulating melanocyte activity by preventing excessive melanin production triggered by UV radiation. Inhibiting melanosomes from transferring melanin from the melanocytes to keratinocytes is another means of reducing the appearance of skin pigmentation. Skin pigmentation is controlled by the amount of melanosomes transferred to the keratinocytes, wherefore by inhibiting this transfer, skin pigmentation will appear to be decreased.

The Portulaca oleracea extract preferably is included in the composition of the present invention in concentrations ranging from 0.002% to 0.03% by weight, based on the total weight of the composition.

UV-filter substances suitable for use in the composition of the invention comprise all substances which have been or currently are approved in sunscreen compositions for use in the United States and elsewhere. Non-limiting examples thereof include para-aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, octisalate, sulisobenzone, trolamine salicylate, diethanolamine methoxycinnamate, digalloyl trioleate, ethyl dihydroxypropyl PABA, glyceryl aminobenzoate, lawsone with dihydroxy acetone, red petrolatum, titanium dioxide, zinc oxide, and the like. Several other sunscreen active ingredients are approved for use in other countries. Non-limiting examples approved outside the U.S. include 4-methylbenzylidene camphor, bis-ethylhexyloxyphenol methoxyphenyl triazine, methylene bis-benzotriazolyl tetramethylbutylphenol, and Polysilicone-15.

It is typical to use combinations of two or more skincare active agents in a cosmetic composition. Preferably, the skincare active agents are present in a concentration that is consistent with the FDA guidelines. The use of a combination of active agents is especially desirable if a sunscreen composition is to achieve higher levels of ultraviolet absorption and/or to provide useful absorption over a wider range of ultraviolet wavelengths than is possible in the case with a single active agent. Preferably, the sunscreen active agent(s) are present in concentrations that are consistent with the FDA sunscreen monograph for sunscreen active agents and are believed to provide the requisite SPF in accordance with the FDA monograph for such sunscreens.

If one or more inorganic and/or organic UV filter substances are present in the composition of the invention, the total concentration thereof preferably ranges from about 1% to about 50% by weight, e.g., from about 5% to about 45% by weight or from about 10% to about 40% by weight, based on the total weight of the composition.

It also has been found that the addition of diethylhexyl syringylidene malonate offers protection against VL and UVA pigmentation in melanin-rich skin. In view thereof, the cosmetic composition of the invention advantageously comprises also diethylhexyl syringylidene malonate, preferably in a concentration of from about 0.1% to about 3% by weight, based on the total weight of the composition.

The cosmetic composition of the invention may optionally include other active or inactive ingredients such as those which are commonly present in cosmetic compositions. Non-limiting examples thereof include cosmetically acceptable carriers, oils, sterols, amino acids, moisturizers, powders, photoactives, colorants (including pigments and/or dyes), pH adjusters, perfumes/fragrances, essential oils, plant extracts, vitamins such as vitamins A, C, and E, essential fatty acids, sphingolipids, self-tanning compounds such as dihydroxyacetone (DHA) and erythrulose, retinol and its derivatives; excipients, fillers, emulsifiers surfactants, film formers, chelating agents, gelling agents, thickeners, emollients, humectants, moisturizers, minerals, viscosity and/or rheology modifiers, keratolytics, retinoids, hormonal compounds, alpha- and beta-hydroxy acids, alpha-keto acids, anti-mycobacterial agents, anti-fungal agents, anti-microbials, anti-virals, analgesics, anti-allergenic agents, antihistamines, anti-inflammatory agents, anti-irritants, anti-neoplastics, immune system boosting agents, immune system suppressing agents, anti-acne agents, anesthetics, antiseptics, insect repellents, skin cooling compounds, skin protectants, skin penetration enhancers, exfoliants, lubricants, fragrances, colorants, staining agents, depigmenting agents, hypopigmenting agents, preservatives, stabilizers, pharmaceutical agents, and mixtures thereof.

It may also be advantageous to include one or more tyrosinase inhibitors in the composition of the invention. A specific example thereof is rucinol, also known as 4-n-butylresorcinol. Other examples of suitable tyrosinase inhibitors include thiamidol and other naturally occurring inhibitors such as, e.g., epigallocatechin gallate, quercitin, arbutin, azelaic acid, ellagic acid, kojic acid, hydroquinone, and aloesin.

The cosmetic composition of the present invention preferably provides a high level of UV-protection to protect skin from the damage caused by the sun, easy applicability and good spreadability.

Also provided by the present invention are compositions with formulation aesthetics that target beauty sector consumers, as well as sunscreen sector consumers. When organic sunscreen actives are used in a composition, the skin feel generally needs to be improved for a lotion aiming at beauty sector consumers. In some embodiments, the composition of the invention is suitable for use as sunscreen lotion and includes ingredients which are carefully selected to improve initial skin feel, skin feel during drying time, and final skin feel.

To sum up, the present invention provides the following items:

1. A cosmetic composition, wherein the composition is formulated for topical application to skin and comprises an extract of Portulaca oleracea in an amount which is effective for reducing pigmentation and skin-darkening in melanin-rich skin.

2. The composition of item 1, wherein the composition comprises from about 0.001% to about 0.5% by weight of the extract of Portulaca oleracea, based on a total weight of the composition.

3. The composition of item 2, wherein the composition comprises from about 0.002% to about 0.03% by weight of the extract of Portulaca oleracea.

4. The composition of any one of the preceding items, wherein the composition further comprises diethylhexyl syringylidene malonate.

5. The composition of item 4, wherein the composition comprises from about 0.1% to about 3% by weight of diethylhexyl syringylidene malonate, based on a total weight of the composition.

6. The composition of any one of the preceding items, wherein the composition further comprises one or more UV filter substances.

7. The composition of item 6, wherein the composition comprises from about 1% to about 50% by weight of the one or more UV filter substances, based on a total weight of the composition.

8. The composition of any one of the preceding items, wherein the composition further comprises at least one of Butyl Methoxydibenzoylmethane, Octocrylene, Homosalate, Ethylhexyl Salicylate, zinc oxide, and titanium dioxide.

9. The composition of any one of the preceding items, wherein the composition is present as a gel, lotion, cream or spray.

10. The composition of any one of the preceding items, wherein the composition is an emulsion.

11. The composition of any one of the preceding items, wherein the composition further comprises a thickener.

12. The composition of any one of the preceding items, wherein the composition further comprises ethanol.

13. The composition of any one of the preceding items, wherein the composition further comprises one or both of glycyrrhetinic acid and Glycyyrhiza Inflata root extract.

14. The composition of any one of the preceding items, wherein the extract of Portulaca oleracea has been obtained by extracting the entire plant of Portulaca oleracea or a part thereof with ethanol and/or water.

15. The composition of any one of the preceding items, wherein the extract of Portulaca oleracea is capable of reducing pigmentation of melanin-rich skin and preventing skin darkening caused by UV radiation and visible light.

16. The composition of any one of the preceding items, wherein the extract of Portulaca oleracea is capable of alleviating or preventing post-inflammatory hyperpigmentation in melanin-rich skin.

17. The composition of any one of the preceding items, wherein the extract of Portulaca oleracea is capable of alleviating or preventing excessive melanin production triggered by UV radiation in melanin-rich skin.

18. A method of reducing pigmentation and skin-darkening in melanin-rich skin, wherein the method comprises topically applying to skin in need thereof the composition of any one of the preceding items in an amount which is effective for reducing pigmentation and skin-darkening in melanin-rich skin.

19. The method of item 18, wherein post-inflammatory hyperpigmentation in melanin-rich skin is alleviated or prevented.

20. The method of item 18, wherein excessive melanin production triggered by UV radiation in melanin-rich skin is alleviated or prevented.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those of skill in the art how the several forms of the present invention may be embodied in practice.

Unless otherwise stated, all weight percentages (% by weight) given below refer to the total weight of the cosmetic composition.

Unless otherwise stated, all tests and measurements were performed under “normal conditions”. The term “normal conditions” refers to 20° C., 1013 hPa and a relative humidity of 50%.

### Example

An example of a suitable sunscreen composition according to the present invention was prepared using methods known to one skilled in the art of formulating cosmetic compositions.

In general, all water-soluble ingredients such as thickeners, chelating agents, humectants, pH adjusters, water dispersible film formers, hydrophilic skin adhesion promoters and/or water-soluble actives are placed in a main vessel This water phase is mixed until uniform and heated to about 80° C.

In a separate vessel, the oil soluble ingredients such as solvents, emollients, emulsifiers, emulsion stabilizers, preservatives, hydrophobic skin adhesion promoters, and/or oil soluble actives, including photoactives, are mixed until uniform and heated to about 80° C.

The oil phase is added to the water phase, homogenized for about 5 minutes until uniform, and the resultant mixture is cooled to room temperature. During the cooling period, the remaining ingredients are admixed. Any ingredients that are heat sensitive are added at this point.

The cosmetic compositions of the present invention can then be formulated and packaged as, for example, a lotion, cream, gel, spray, including alcohol-based sprays, or other acceptable forms for a cosmetic composition, in particular, a sunscreen composition.

The sunscreen composition was subsequently subjected to a clinical test.

Components of exemplary compositions are listed in TABLE 1 and TABLE 2 below.

TABLE 1

| Composition Example No. | 1 | 2 | 3 |
|---|---|---|---|
| Dibutyl Adipate | 2.00 | 2.00 | 2.00 |
| C12-15 Alkyl Benzoate | 1.00 | 1.00 | 1.00 |
| Polyamide-8 | 1.50 | 1.50 | 1.50 |
| Arachidyl Alcohol + Behenyl | 0.32 | 0.32 | 0.32 |
| Alcohol (&) Arachidyl Glucoside | | | |
| Acrylates/C10-30 Alkyl Acrylate | 0.25 | 0.25 | 0.25 |
| Crosspolymer | | | |
| Sodium Stearoyl Glutamate | 0.50 | 0.50 | 0.50 |
| Butyl Methoxydibenzoylmethane | — | 3.00 | 3.00 |
| Octocrylene | — | 10.00 | 10.00 |
| Homosalate | — | 15.00 | 15.00 |
| Ethylhexyl Salicylate | — | 5.00 | 5.00 |
| Bis-Ethylhexyloxyphenol | — | 3.00 | 3.00 |
| Methoxyphenyl Triazine | | | |
| Diethylhexyl Syringylidene | — | — | 1.00 |
| malonate | | | |
| Portulaca Oleracea Extract | — | — | 5.00 |
| Glycereth-26 | 2.00 | 2.00 | 2.00 |
| Glycerin | 2.00 | 2.00 | 2.00 |
| Xanthan Gum | 0.20 | 0.20 | 0.20 |
| Potassium Hydroxide | 0.14 | 0.14 | 0.14 |
| Disodium EDTA | 0.05 | 0.05 | 0.05 |
| Ethylhexylglycerin | 0.20 | 0.20 | 0.20 |
| Phenoxyethanol | 0.80 | 0.80 | 0.80 |
| Ethanol | 6.00 | 6.00 | 6.00 |
| Water | q.s. | q.s. | q.s. |
| | to 100 | to 100 | to 100 |
| ISO 23698 DRS UVA-PF | 1.20 | 25.0 | 36.8 |

TABLE 2

| Composition Example No. | 4 | 5 | 6 |
|---|---|---|---|
| Triheptanoin + Coco-caprylate/caprate + | 4.00 | 4.00 | 4.00 |
| Dilinoleic Acid/Butanediol Copolymer + | | | |
| Castor Oil/IPDI Copolymer | | | |
| Dibutyl Adipate | 4.00 | 4.00 | 4.00 |
| Dicaprylyl Carbonate + Stearalkonium | 4.00 | 4.00 | 4.00 |
| Hectorite + Propylene Carbonate | | | |
| Dicaprylyl Carbonate | 4.00 | 4.00 | 4.00 |
| C12-15 Alkyl Benzoate | 3.00 | 3.00 | 3.00 |
| Neopentyl Glycol Diheptanoate | 5.00 | 4.00 | 4.00 |
| Butylene Glycol Dicaprylate/Dicaprate | 5.00 | 4.75 | 3.25 |
| Caprylic/Capric Triglyceride | 3.00 | 3.00 | 3.00 |
| Ethylhexyl Methoxycrylene | 2.00 | 2.00 | 2.00 |
| PPG-15 Stearyl Ether | 2.00 | 2.00 | 2.00 |
| Dimethicone | 1.00 | 1.00 | 1.00 |
| Bis-Octyldodecyl Dimer | 2.00 | 2.00 | 2.00 |
| Dilinoleate/Propanediol Copolymer | | | |
| Maleated Soybean Oil | 1.00 | 1.00 | 1.00 |
| Glyceryl/Octyldodecanol Esters | | | |
| Polyhydroxystearic Acid + Caprylic/Capric | 1.00 | 1.00 | 1.00 |
| Triglyceride + Isostearic Acid +) Lecithin + | | | |
| Polyglyceryl-3 Polyricinoleate | | | |
| Polyglyceryl-4 Diisostearate/ | 3.00 | 3.00 | 3.00 |
| Polyhydroxystearate/Sebacate + | | | |
| Caprylic/Capric Triglyceride + Polyglyceryl-3 | | | |
| Oleate + Diisostearoyl Polyglyceryl-3 Dimer | | | |
| Dilinoleate | | | |
| Hydroxyacetophenone | 0.30 | 0.30 | 0.30 |
| Beeswax | 0.50 | 0.50 | 0.50 |
| Dimethicone | 1.00 | 1.00 | 1.00 |
| Zinc Oxide + Triethoxycaprylylsilane | 25.00 | 25.00 | 25.00 |
| Water | 19.00 | 18.84 | 15.75 |
| Butylene Glycol | 2.00 | 2.00 | 2.00 |
| 1,2-Hexanediol | 0.80 | 0.80 | 0.80 |
| Sodium Chloride | 0.40 | 0.40 | 0.40 |
| Diethylhexyl Syringylidenemalonate | — | 1.00 | 1.00 |
| Portulaca Oleracea Extract* | — | — | 5.00 |
| Tocopherol | — | 0.25 | — |
| Sodium Ascorbyl Phosphate | — | 0.01 | — |
| Glycyrrhetinic Acid | — | 0.10 | — |
| Glycyrrhiza Inflata Root Extract | — | 0.05 | — |
| Ethanol | 4.00 | 4.00 | 4.00 |
| Silica | 3.00 | 3.00 | 3.00 |
| ISO 23698 DRS UVA-PF | 24.7 | 21.2 | 27.1 |

Composition No. 1 is a base formula without any UV filters, photostabilizer, and Portulaca oleracea extract. DRS UVA protection values were evaluated on the mineral (ZnO) based sunscreens (see TABLE 2). The addition of the combination of UV filters, photostabilizer, and Portulaca oleracea extract led to an increased in-vitro UVA protection compared to the rest of the compositions. Any one or more of the Portulaca oleracea extract may be used as shown in TABLE 3. However, the effectiveness of the extract is dependent on the solvent and method used for extraction. As in the exemplary composition shown in TABLE 1 and TABLE 2, the Portulaca oleracea extract used in the final composition is EverExtract™ PO as referenced in TABLE 3. This extract is obtained by extraction of the plant or parts thereof with ethanol and is present as a solution in water and butylene glycol.

TABLE 3

| | Trade | INCI | concentration |
|---|---|---|---|
| Vendor | Name | Name | of extract |
| Acme Hardesty | Portulaca Oleracea | Glycerin, water, Portulaca | not available |
| Co./Green | Extract | oleracea extract | |
| Mountain Biotech | | | |
| Easthill | Advanced | Propanediol (and) Water | not available |
| Corporation | Portulaca Oleracea | (and) Portulaca Oleracea | |
| | Extract | Extract (and) 1,2-Hexanediol | |
| The Garden of | Portulaca Extract- | Portulaca oleracea extract | not available |
| Naturalsolution | TLS | | |
| Co., Ltd. (former | | | |
| Natural Solution | | | |
| Co.,Ltd.) | | | |
| Vital-Chem Zhuhai | Portulaca Extract- | Portulaca oleracea extract | 1-5% |
| Co.,Ltd. | Liquid | | |
| KimiKa, LLC | iActive Portulaca | Portulaca oleracea extract | not available |
| (Formerly | | | |
| Cosphatech) | | | |
| Paradigm Science | Portulaca Extract | Water (and) Glycerin (and) | 0.1-1% |
| Inc. | GN | Portulaca Oleracea Extract | |
| Paradigm Science | Portulaca Extract | Water (and) Butylene Glycol | 0.1-1% |
| Inc. | | (and) Portulaca Oleracea | |
| | | Extract | |
| JAKA Biotech Co., | Gerbex ® Portulaca | Glycerin (and) Water (and) | not available |
| Ltd. | | Portulaca Oleracea Extract | |
| Univar Solutions | Purslane Extract | Portulaca oleracea extract | not available |
| | in Water | | |
| Sino Lion* | EverExtractTM PO | Water (and) Butylene Glycol | 0.2%-0.6% |
| | | (and) Portulaca oleracea | |
| | | extract | |
| Hyundai Bioland | Portulaca Extract | Water (and) Butylene Glycol | 0.1-1% |
| Co., Ltd. | | (and) Portulaca Oleracea | |
| | | Extract | |
| DKSH North | | Aqua (and) Portulaca | not available |
| America, Inc | | Oleracea Extract (and) Citric | |
| | | Acid (and) Sodium Benzoate | |
| | | (and) Potassium Sorbate | |

### Clinical Study

Ten volunteers with melanin-rich skin (Fitzpatrick skin types IV, V, and VI) were included in the clinical study. A dose of 380 J/cm² was determined to be sufficient to induce detectable erythema and pigmentation in subjects with Fitzpatrick skin phototypes VI, V, and VI by the solar simulator whose characteristics are set forth in TABLE 4 below. This unique light source was built to simulate full sunlight similar to Matchu Pitchu, simulating high UVB and UVA balance to expedite the development of erythema in skin. After determining the effective dose that induces significant erythema and pigmentation on skin, 3 days prior to the exposure the tested compositions were applied every day on randomized areas on the backs of the 10 volunteers at an optical density of 2 mg/cm². Then, all tested compositions were applied at 2 mg/cm² on the subjects and thereafter the sites treated with the composition were exposed to 380 J/cm² with the solar simulator for the required time. One site was untreated and referred to as unprotected exposed site. The assessments were taken using Diffuse Reflectance Spectroscopy (DRS) for oxyhemoglobin, melanin assessment and Area Under the Curve (AUC) for discoloration, clinical grading (for erythema and pigmentation) and digital cross-polarized images from each of the treated and untreated sites immediately, 24 hr, and 7 days after exposure.

### Discussion of Test Results

When skin was exposed to visible light included in the clinical study the immediate response of the skin was an erythema response which lasted for about 2 hours after the exposure. This skin response has already been documented in light or dark skin individuals. In the case of dark skin complexion, new pigmentation is generated 7 days after the exposure. Antioxidants have been shown to be effective in suppressing this initial skin erythema and also the pigmentation in dark skin.

As shown in TABLE 1, the DRS-UVA assessment illustrates that the composition comprising UV filters, Portulaca oleracea extract, and diethylhexyl syringylidene malonate provided an increased in-vitro UVA protection when compared to compositions without said ingredients.

The use of Portulaca oleracea extract was shown to be effective in suppressing this initial erythema and pigmentation in melanin-rich skin against VL and UVA as shown in Table 3. The results also showed significantly lower pigmentation compared with untreated but irradiated skin at the different time points.

The results of AUC for the treated sites 7 days after exposure showed more pigmentation and skin darkening of the treated sites versus unexposed sites as shown in Table 3.

Overall, the composition comprising of Portulaca oleracea extract significantly reduced the initial erythema and pigmentation of melanin-rich skin, contributing to protection against VL and UVA.

The solar simulator mentioned above and used for testing was developed to simulate solar radiation (Solar Light Company, LLC, Glenside, Pennsylvania, model LS1000-4S-UVA-VI) including wavelengths from 290 nm up to 750 nm, continuously and simulating solar radiation Air Mass 1.5 (the solar radiation close to Machu Pitchu, Peru). The radiometric characteristics of the solar simulator are illustrated in TABLE 4 below.

TABLE 3
Mean Oxyhemoglobin (exposed-control)
and mean AUC (exposed-control)

| | | |
|---|---|---|
| Mean Oxyhemoglobin | Mean AUC | |
| (exposed -control) | (exposed - control) | Note |

Chemical UV filters

| | | |
|---|---|---|
| 0.473833 | 107.623 | control |
| 0.436526 | 78.529 | UV filters only |
| 0.413507 | 77.754 | UV filters + 5% PO |

Inorganic UV filters

| | | |
|---|---|---|
| 0.435074 | 78.803 | UV filters only |
| 0.412723 | 73.071 | UV filters + 5% PO |

TABLE 4
Characteristics of UV source used
for spectroradiometric measurements

UV source (Power)
LS1000-4S-UVA-VIS
1215 W

| | | AM1.5 |
|---|---|---|
| Energetic Irradiance | Unit | Center |
| 250-280 nm | W · m − 2 | 0.00 |
| <290 nm | W · m − 2 | 0.02 |
| UVB (290-320 nm) | W · m − 2 | 0.19 |
| UVA (320-400 nm) | W · m − 2 | 63.2 |
| UVA-2 (320-340 nm) | W · m − 2 | 0.7 |
| UVA-1 (340-400 nm) | W · m − 2 | 62.5 |
| Total UV (290-400 nm) | W · m − 2 | 63.4 |
| Visible (400-780 nm) | W · m − 2 | 913.16 |
| NIR (780-1600 nm) | W · m − 2 | 85.040 |
| UV + VIS (290-780 nm) | W · m − 2 | 976.5 |
| Total (UV + VIS + NIR) | W · m − 2 | 1061.6 |
| Ratio UVA/UVB | Ratio | 333.54 |
| Ratio UVA1/UVA2 | Ratio | 92.67 |
| Ratio VIS + IR/Total | Ratio | 94.03% |
| Total irrad. | W · m − 2 | 976.5 |
| (UV + Vis < 200) | | |
| UVA2 portion (≥20%) | % | 1.1% |
| UVA1 portion (≥60%) | % | 98.9% |

What is claimed is:

1. A cosmetic composition, wherein the composition is formulated for topical application to skin and comprises at least one extract of Portulaca oleracea in a concentration which is effective for reducing pigmentation and skin-darkening in melanin-rich skin.

2. The composition of claim 1, wherein the composition comprises from 0.001% to 0.5% by weight of the at least one extract of Portulaca oleracea, based on a total weight of the composition.

3. The composition of claim 2, wherein the composition comprises from 0.002% to 0.03% by weight of the at least one extract of Portulaca oleracea.

4. The composition of claim 1, wherein the composition further comprises diethylhexyl syringylidene malonate.

5. The composition of claim 4, wherein the composition comprises from about 0.1% to about 3% by weight of diethylhexyl syringylidene malonate, based on a total weight of the composition.

6. The composition of claim 1, wherein the composition further comprises one or more UV filter substances.

7. The composition of claim 6, wherein the composition comprises from about 1% to about 50% by weight of the one or more UV filter substances, based on a total weight of the composition.

8. The composition of claim 1, wherein the composition further comprises at least one of butyl methoxydibenzoylmethane, octocrylene, homosalate, ethylhexyl salicylate, zinc oxide, and titanium dioxide.

9. The composition of claim 1, wherein the composition is present as a gel, lotion, cream or spray.

10. The composition of claim 1, wherein the composition is present as an emulsion.

11. The composition of claim 1, wherein the composition further comprises a thickener.

12. The composition of claim 1, wherein the composition further comprises ethanol.

13. The composition of claim 1, wherein the composition further comprises one or both of glycyrrhetinic acid and Glycyyrhiza Inflata root extract.

14. The composition of claim 1, wherein the at least one extract of Portulaca oleracea has been obtained by extracting the entire plant of Portulaca oleracea or a part thereof with ethanol and/or water.

15. The composition of claim 1, wherein the at least one extract of Portulaca oleracea is capable of reducing pigmentation of melanin-rich skin and preventing skin darkening caused by UV radiation and visible light.

16. The composition of claim 1, wherein the at least one extract of Portulaca oleracea is capable of alleviating or preventing post-inflammatory hyperpigmentation in melanin-rich skin.

17. The composition of claim 1, wherein the at least one extract of Portulaca oleracea is capable of alleviating or preventing excessive melanin production triggered by UV radiation in melanin-rich skin.

18. A method of reducing pigmentation and skin-darkening in melanin-rich skin, wherein the method comprises topically applying to skin in need thereof the composition of claim 1 in an amount which is effective for reducing pigmentation and skin-darkening in melanin-rich skin.

19. The method of claim 18, wherein post-inflammatory hyperpigmentation in melanin-rich skin is alleviated or prevented.

20. The method of claim 18, wherein excessive melanin production triggered by UV radiation in melanin-rich skin is alleviated or prevented.
